(19) United States
(12) Patent Application Publication
(10) Pub. No.: US 2002/0037301 A1
(43) Pub. Date: Mar. 28, 2002

(54) Film-forming cosmetic composition

(76) Inventor: Valerie De La Poterie, Le Chatelet en Brie (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/880,868

(22) Filed: Jun. 15, 2001

(30) Foreign Application Priority Data
Jun. 15, 2000 (FR) 00 07655

(51) Int. Cl. A61K 7/00

(52) U.S. Cl. 424/401

(57) ABSTRACT

A cosmetic composition containing an aqueous dispersion of particles of at least one free-radical film-forming polymer and an aqueous dispersion of at least one polymer having at least one fatty chain, for example an alkyd resin, having a length in terms of oil ranging from 5% to 90%, wherein when the weight ratio of the at least one free-radical film-forming polymer and the at least one polymer having at least one fatty chain is less than 1.5:1, the at least one polymer having at least one fatty chain has a length in terms of oil of less than 50%, and wherein when the weight ratio is greater than or equal to 1.5:1, the at least one free-radical film-forming polymer has a glass transition temperature (Tg) of less than or equal to 30° C. A method for treating keratinous material is also provided.

The present invention relates to a film-forming cosmetic composition comprising an aqueous dispersion of particles of at least one free-radical film-forming polymer and an aqueous dispersion of at least one polymer having at least one fatty chain. The cosmetic composition may be used, for example, as a make-up or care composition for keratinous material such as the skin, lips, nails, eyelashes, eyebrows, and hair of human beings. The present invention relates to a use of this composition to treat and care for keratinous material, for example, the treatment and the care of the nails.

In addition, the present invention relates to a composition containing at least one free-radical film-forming polymer, which is capable of forming a homogeneous and continuous film on a support such as the nails, eyelashes, and hair.

Among the characteristics which the film-forming cosmetic composition, for example, a nail varnish, should possess are: production of a homogeneous film having a degree of flexibility, and good resistance of the film, for example, so as to avoid cracking and scaling of the polymer film.

The film-forming cosmetic composition comprising the at least one free-radical film-forming polymer in aqueous dispersion is currently undergoing rapid development, for example, as make-up products, so as to replace compositions in solvent medium, for example, solvent nail varnishes. This is not only for safety reasons for the consumer, but also for environmental reasons because of the absence of volatile compounds in such compositions.

These film-forming cosmetic compositions in aqueous medium may comprise a film-forming polymer forming a film at room temperature, but some polymers lead to the formation of a soft and sticky film which does not allow the production of a resistant film. In other cases, these film-forming cosmetic compositions may comprise a film-forming polymer having a high transition temperature (greater than room temperature, that is 25° C.) and it is then necessary to combine with this polymer at least one of a plasticizing agent and a coalescing agent in order to obtain a film at room temperature exhibiting satisfactory properties. Thus, U.S. Pat. No. 4,158,053 describes the use of glycol ethers as coalescing agents in nail varnish compositions containing acrylic polymers in aqueous dispersion. However, these glycol ethers exhibit toxicological risks, thereby limiting and forbidding their use in these compositions. Likewise, the need to limit the use of cosolvents with the aim of preparing the products without volatile organic compounds (termed non-VOC products), does not make it possible to obtain films having the desired optimum properties. There is thus a need to have available other compounds, other than cosolvents, which can act as a plasticizer for the film.

An aim of the present invention is to provide a film-forming composition, well suited to use as a make-up for and/or for the care of keratinous material, which has good cosmetic properties and does not have at least one of the disadvantages mentioned above.

The Inventor has discovered a composition comprising an aqueous dispersion of at least one free-radical film-forming polymer and at least one polymer having at least one fatty chain. The combination of these polymers according to the present invention may lead to the formation of a film which is resistant and thus may avoid the appearance of cracks, without using a cosolvent.

Document EP-A-418469 describes a nail varnish comprising an aqueous dispersion of a film-forming polymer and an alkyd resin.

A subject of the present invention is a cosmetic composition comprising an aqueous dispersion of particles of at least one free-radical film-forming polymer and an aqueous dispersion of at least one polymer having at least one fatty chain and having a length in terms of oil ranging from 5% to 90%, wherein when the weight ratio of the at least one free-radical film-forming polymer and the at least one polymer having at least one fatty chain is less than 1.5:1, the at least one polymer having at least one fatty chain has a length in terms of oil of less than 50%; and wherein when the weight ratio is greater than or equal to 1.5:1, the at least one free-radical film-forming polymer has a glass transition temperature (Tg) of less than or equal to 30° C.

Another subject of the invention is at least one of a nail varnish and a nail care product comprising a composition as defined above.

Another subject of the invention is a method for cosmetically treating and applying make-up to keratinous material, comprising applying to the keratinous material a composition as defined above.

Another subject of the invention is the use of an aqueous dispersion of particles of at least one free-radical film-forming polymer and an aqueous dispersion of at least one polymer having at least one fatty chain as defined above for the production of a film.

Another subject of the invention is the use of an aqueous dispersion of at least one polymer having at least one fatty chain in a composition as defined above as a plasticizing agent for the at least one free-radical film-forming polymer.

The cosmetic composition according to the present invention comprises a cosmetically acceptable medium which is a medium compatible with keratinous material.

The at least one polymer having at least one fatty chain used according to the present invention may be a polycondensate, for example, a polycondensate chosen from polyesters, polyesteramides, polyurethanes, and polyureas. The at least one polymer having at least one fatty chain may be an alkyd resin. The term “fatty chain” should be understood to indicate a hydrocarbon chain comprising at least 8 carbon atoms, for example, from 8 to 46 carbon atoms. The term “length in terms of oil” of at least one polymer having at least one fatty chain should be understood to indicate the percentage by weight of fatty chain in the polymer.

The alkyd resin used according to the present invention may be a polyester comprising hydrocarbon chains of fatty acids. Such a resin is, for example, described in Encyclopedia of Chemical Technology, Kirk-Othmer, 4th edition, volume 2, pages 53 to 63, which is incorporated by reference herein. These resins may be obtained by polymerizing polyols and polyacids and their corresponding anhydrides, in the presence of fatty acids. These fatty acids may be used as they are, in the form of fatty acid triglycerides, and they may be present in the oils during the synthesis of the alkyd resin.

Because of the presence of hydrocarbon chains of fatty acids in the alkyd resin, alkyd resins are commonly defined by their length in terms of oil. The term “length in terms of oil” of an alkyd resin should be understood to indicate the percentage by weight of hydrocarbon chain of fatty acid present in the alkyd resin.

As polyols which may be used for the synthesis of an alkyd resin, there may be mentioned, for example, glycerol, pentaerythritol, trimethylolpropane, trimethylolethane, neopentyl glycol, propylene glycol, ethylene glycol, 1,6-hexanediol, 1,4-butanediol, and diethylene glycol.

As polyacids and their corresponding anhydrides, for example, phthalic anhydride, isophthalic acid, terephthalic acid, trimellitic anhydride, maleic anhydride, adipic acid, fumaric acid, azelaic acid, and sebacic acid may be mentioned. The fatty acids may correspond to the formula R—COOH, wherein R may be chosen from saturated and unsaturated hydrocarbon groups having, for example, from 7 to 45 carbon atoms, as a further example, from 9 to 35 carbon atoms, as a further example, from 15 to 35 carbon atoms, and as a further example, from 15 to 21 carbon atoms.

As fatty acids, for example, palmitic acid, stearic acid, oleic acid, ricinoleic acid, linoleic acid, and linolenic acid may be mentioned.

Fatty acids are present in the majority of oils of natural origin, for example, in the form of triglycerides. Fatty acid triglycerides may be esters resulting from the reaction of the three alcohol functional groups of glycerol with fatty acids wherein it may be possible for these fatty acids to be identical or different. Oils of natural origin can, therefore, be used in the polymerization. They may be chosen from linseed oil, Chinese wood oil, oiticica oil, soya-bean oil, sunflower oil, safflower oil, castor oil, coconut (copra) oil, olive oil, palm oil, rapeseed oil, groundnut oil, and tallol (also called tall oil).

The at least one polymer having at least one fatty chain, for example, an alkyd resin, in the composition according to the present invention may be present in the composition in the form of an aqueous dispersion and may be, therefore, insoluble in water (its solubility in water at 25° C. being less than 1% by weight).

As alkyd resins which can be used according to the present invention, for example, those sold under the names NECOWEL 581® (50% in soya-bean oil), NECOWEL 585® (20% in sunflower oil), NECOWEL 580® (20% in oil), NECOWEL 586 N® (50% in soya-bean oil), NECOWEL EP 1161® (50% in soya-bean oil), NECOWEL EP 1213® (20% in oil), NECOWEL EP 2009® (32% in sunflower oil), NECOWEL EP 2019® (20% in oil), NECOWEL EP 2275® (35% in oil), NECOWEL EP 2329® (34% in oil) and NECOWEL EP 3016® (30% in oil) by the company ASHLAND, and URADIL XP 515 AZ® (73% in tallol) and URADIL XP 516 AZ® (63% in tallol) by the company DSM RESINS may be mentioned.

In the composition according to the present invention, the at least one polymer having at least one fatty chain may be present in the composition in an amount ranging from 0.5% to 40% by weight relative to the total weight of the composition, for example from 2% to 30% by weight, and as a further example from 5% to 20% by weight relative to the total weight of the composition.

The at least one free-radical film-forming polymer according to the present invention may be chosen from vinyl polymers and copolymers, for example, acrylic polymers.

Vinyl polymers may result from the polymerization of monomers with ethylenic unsaturation having at least one of at least one acidic group, esters of these acidic monomers, and amides of these acidic monomers.

Anionic free-radical film-forming polymers, resulting from the polymerization of monomers having at least one acidic group, for example, carboxylic acid group may be used.

As monomers carrying an acidic group, there may be used α,β-ethylenically unsaturated carboxylic acids chosen from acrylic acid, methacrylic acid, crotonic acid, maleic acid, and itaconic acid. For example, (meth)acrylic acid and crotonic acid, and as a further example (meth)acrylic acid may be used.

The esters of acidic monomers may be chosen from the esters of (meth)acrylic acid (also called (meth)acrylates), for example alkyl (meth)acrylates, a further example (C₁ to C₂₀) alkyl (meth)acrylates, and as a further example (C₁ to C₈) alkyl (meth)acrylates; aryl, for example (C₆ to C₁₀) aryl, (meth)acrylates; hydroxyalkyl, for example (C₂ to C₆) hydroxyalkyl, (meth)acrylates.

Among the alkyl (meth)acrylates, methyl methacrylate, ethyl methacrylate, butyl methacrylate, isobutyl methacrylate, 2-ethylhexyl methacrylate, and lauryl methacrylate may be mentioned.

Among the hydroxyalkyl (meth)acrylates, hydroxyethyl acrylate, 2-hydroxypropyl acrylate, hydroxyethyl methacrylate, and 2-hydroxypropyl methacrylate may be mentioned.

Among the aryl (meth)acrylates, benzyl acrylate, and phenyl acrylate may be mentioned.

The (meth)acrylic acid esters may be, for example, alkyl (meth)acrylates.

According to the present invention, the alkyl group of the esters may be chosen from fluorinated and perfluorinated alkyl groups. That is, at least a portion of the hydrogen atoms of the alkyl group may be substituted by fluorine atoms.

As amides of the acidic monomers, for example, (meth)acrylamides, for example, N-alkyl (meth)acrylamides, for example a (C₂ to C₁₂) alkyl may be mentioned. Among the N-alkyl (meth)acrylamides, N-ethyl acrylamide, N-t-butyl acrylamide, and N-t-octyl acrylamide may be mentioned.

The vinyl polymers may result from the homopolymerization or the copolymerization of monomers chosen from vinyl esters and styrene monomers. For example, these monomers may be polymerized with at least one of acidic monomers, their esters, and their amides, such as those mentioned above.

As examples of vinyl esters, vinyl acetate, vinyl neodecanoate, vinyl pivalate, vinyl benzoate, and vinyl tert-butyl benzoate may be mentioned.

As styrene monomers, styrene and alpha-methylstyrene may be mentioned.

The list of monomers given is not limiting and it is possible to use, for example, any monomer known to persons skilled in the art in the categories of acrylic and vinyl monomers (including the monomers chosen from those modified by a silicone and fluorinated chain).

The size of the polymer particles in aqueous dispersion may range, for example, from 10 to 500 nm, and as a further example may range from 20 to 300 nm.

As acrylic polymers which can be used according to the present invention, those sold under the names NEOCRYL XK-90®, NEOCRYL A-1070®, NEOCRYL BT-62®, NEOCRYL A-1079®, NEOCRYL A-523®, NEOCRYL XK-52® and NEOCRYL A-639® by the company ZENECA, DOW LATEX 432® by the company DOW CHEMICAL, and CARBOSET CR 712® and CARBOSET CR 714® by the company GOODRICH may be mentioned.

In the composition according to the present invention, the at least one free-radical film-forming polymer may be present in the composition in an amount ranging from 3% to 50% by weight relative to the total weight of the composition, and for example ranging from 10% to 35% by weight.

According to the present invention, when the weight ratio of the at least one free-radical film-forming polymer and the at least one polymer having at least one fatty chain is less than 1.5:1, the at least one the polymer having at least one fatty chain has a length in terms of oil of less than 35%. Moreover, when the weight ratio of the at least one free-radical film-forming polymer and the at least one polymer having at least one fatty chain is greater than or equal to 1.5:1, the at least one free-radical film-forming polymer has a glass transition temperature (Tg) ranging from −40° C. to 30° C., for example the Tg may be less than or equal to 25° C., and may range, for example, from −10° C. to 25° C.

The measurement of the glass transition temperature is carried out by DSC (Differential Scanning Calorimetry) according to the ASTM D3418-97 standard, the disclosure of which is hereby incorporated by reference.

In addition, the composition according to the invention may comprise at least one adjuvant commonly used in cosmetic compositions. Examples of the at least one adjuvant may be chosen from colorants, pigments, pearlescent agents, lacquers, anti-UV agents, thickening agents, surfactants, waxes, silicones, perfumes, preservatives, moisturizing agents, and active agents chosen from D-panthenol, phytantriol, vitamins, biotin, and trace elements. Of course, persons skilled in the art will be careful to choose this or these optional adjuvants and/or their quantity, such that the properties of the composition according to the present invention are not, or not substantially, adversely affected by the addition envisaged.

The composition according to the present invention may be used for the application of make-up to and cosmetic treatment of keratinous material. The make-up composition may be, for example, a nail varnish, a nail care composition, an eyeliner, a mascara, a foundation, an eyeshadow, a blusher, a lipstick, a concealer, or a make-up product for the body such as a temporary or semi-permanent tattoo product. The composition for cosmetic treatment may be a care composition for the face, the neck, the hands, and the body. It may also constitute an anti-sun or a self-tanning composition.

Unless otherwise indicated, all numbers expressing quantities of ingredients, properties such as molecular weight, reaction conditions, and so forth used in the specification and claims are to be understood as being modified in all instances by the term “about”. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. The examples which follow are intended to illustrate the invention without, however, limiting its scope.

### EXAMPLE 1

An eyeliner having the following composition was prepared:

| | | |
|---|---|---|
| Aqueous dispersion of acrylic polymer | 16.1 | g |
| containing 46.6% of active material | | |
| (NEOCRYL A-639 ® from the company | | |
| ZENECA) | | |
| Aqueous emulsion of alkyd resin at 20% | 39.8 | g |
| in oil and at 44% in water (NECOWEL | | |
| 585 ® from the company ASHLAND) | | |
| black iron oxide | 15 | g |
| propylene glycol | 15 | g |
| water | qs 100 | g |

The composition formed a smooth and homogeneous film after application to the edge of the eyelids.

### EXAMPLE 2

A product for the lips having the following composition was prepared:

| | | |
|---|---|---|
| Aqueous dispersion of acrylic polymer at | 34.1 | g |
| 41% in water (CARBOSET CR 712 ® | | |
| from the company GOODRICH) | | |
| Aqueous emulsion of alkyd resin at 50% | 12.8 | g |
| in oil and at 47% in water (NECOWEL | | |
| 581 ® from the company ASHLAND) | | |
| Pigments | 4 | g |
| Glycerine | 2.5 | g |
| Water | qs 100 | g |

The composition was easily applied to the lips and formed thereon a smooth and glossy film.

### EXAMPLE 3

A nail varnish having the following composition was prepared:

| | | |
|---|---|---|
| Aqueous dispersion of acrylic polymer | 32.2 | g |
| containing 46.6% of active material | | |
| (NEOCRYL A 639 ® from the company | | |
| ZENECA) | | |
| Aqueous emulsion of alkyd resin at 20% | 34.1 | g |
| in oil and at 44% in water (NECOWEL | | |
| 585 ® from the company ASHLAND) | | |
| Pigments | 3 | g |
| Thickener | 0.3 | g |
| Glycerine | 1.9 | g |
| Water | qs 100 | g |

The varnish was easily applied to the nail and left a resistant and homogeneous film after drying.

### EXAMPLE 4 to 31

Mixtures of an aqueous dispersion of an acrylic polymer and an alkyd resin emulsion were prepared and it was examined whether the mixture formed a film.

The following results were obtained:

| Acrylic/ | | | | |
|---|---|---|---|---|
| alkyd | | | | Film |
| proportion | Alkyd resin | Acrylic polymer | Example | formation |
| 50/50 | Necowel 585 ® | Carboset CR | 4 | Yes |
| | | 712 ® | | |
| | | Carboset CR | 5 | Yes |
| | | 714 ® | | |
| | | Neocryl A 639 ® | 6 | Yes |
| | | Neocryl XK 52 ® | 7 | Yes |
| | Necowel 581 ® | Carboset CR | 8* | No |
| | | 712 ® | | |
| | | Carboset CR | 9* | No |
| | | 714 ® | | |
| | | Neocryl A 639 ® | 10* | No |
| | | Neocryl XK 52 ® | 11* | No |
| | Uradil XP | Carboset CR | 12* | No |
| | 515 AZ ® | 712 ® | | |
| | | Carboset CR | 13* | No |
| | | 714 ® | | |
| | | Neocryl A 639 ® | 14* | No |
| | | Neocryl XK 52 ® | 15* | No |
| 70/30 | Necowel 585 ® | Carboset CR | 16 | Yes |
| | | 712 ® | | |
| | | Carboset CR | 17* | No |
| | | 714 ® | | |
| | | Neocryl A 639 ® | 18* | No |
| | | Neocryl XK 52 ® | 19* | No |
| | Necowel 581 ® | Carboset CR | 20 | Yes |
| | | 712 ® | | |
| | | Carboset CR | 21* | No |
| | | 714 ® | | |
| | | Neocryl A 639 ® | 22* | No |
| | | Neocryl XK 52 ® | 23* | No |
| | Uradil XP | Carboset CR | 24 | Yes |
| | 515 AZ ® | 712 ® | | |
| | | Carboset CR | 25* | No |
| | | 714 ® | | |
| | | Neocryl A 639 ® | 26* | No |
| | | Neocryl XK 52 ® | 27* | No |
| 30/70 | Necowel 585 ® | Carboset CR | 28 | Yes |
| | | 712 ® | | |
| | | Carboset CR | 29 | Yes |
| | | 714 ® | | |
| | | Neocryl A 639 ® | 30 | Yes |
| | | Neocryl XK 52 ® | 31 | Yes |

*Comparative Example

The acrylic/alkyd proportion corresponds to the weight ratio of the at least one free-radical film-forming polymer active material and the alkyd resin.

The total weight of the acrylic polymer and the alkyd resin was 28 g per 100 g of composition and the balance was water.

NECOWEL 585®: an alkyd resin with 20% of sunflower oil sold by the company ASHLAND; NECOWEL 581®: an alkyd resin with 50% of soya-bean oil sold by the company ASHLAND; URADIL XP 515 AZ®: an alkyd resin with 73% of tallow oil sold by the company DSM RESINS; CARBOSET CR 712®: an acrylic-styrene copolymer in aqueous emulsion at 40% sold by the company GOODRICH (Tg=18° C.); CARBOSET CR 714®: an acrylic-styrene copolymer in aqueous emulsion at 42.5% sold by the company GOODRICH (Tg=35° C.); NEOCRYL A 639®: an acrylic-styrene copolymer in aqueous emulsion at 45% sold by the company ZENECA (Tg=67° C.); NEOCRYL XK 52®: an acrylic copolymer in aqueous emulsion at 40% sold by the company ZENECA (glass transition temperature (Tg)>100° C.).

It was observed that, when the weight ratio of the acrylic film-forming polymer and the alkyd resin was greater than 1.5 (acrylic/alkyd ratio=70/30), a film was obtained when the acrylic polymer had a glass transition temperature of less than or equal to 30° C. (Examples 16, 20 and 24).

When the weight ratio of the acrylic film-forming polymer and the alkyd resin was less than 1.5 (acrylic/alkyd ratio=50/50 or 30/70), a film was obtained when the length in terms of oil of the alkyd resin was less than 50% (Examples 4 to 7).

What is claimed is:

1. A cosmetic composition comprising,
an aqueous dispersion of particles of at least one free-radical film-forming polymer and
an aqueous dispersion of at least one polymer having at least one fatty chain,
wherein the at least one polymer having at least one fatty chain has a length in terms of oil ranging from 5% to 90%, and
when the free-radical film-forming polymer/polymer having at least one fatty chain weight ratio is less than 1.5:1, the at least one polymer having at least one fatty chain has a length in terms of oil of less than 50%;
when the free-radical film-forming polymer/polymer having at least one fatty chain weight ratio is greater than or equal to 1.5:1, the at least one free-radical film-forming polymer has a glass transition temperature of less than or equal to 30° C.

2. The composition according to claim 1, wherein when the weight ratio is less than 1.5:1, the at least one polymer having at least one fatty chain has a length in terms of oil of less than 35%.

3. The composition according to claim 1, wherein when the weight ratio is greater than or equal to 1.5:1, the at least one free-radical film-forming polymer has a glass transition temperature (Tg) of less than or equal to 25° C.

4. The composition according to claim 1, wherein the at least one polymer having at least one fatty chain is an alkyd resin.

5. The composition according to claim 1, wherein the at least one free-radical film-forming polymer is present in the composition in an amount ranging from 3% to 50% by weight relative to the total weight of the composition.

6. The composition according to claim 5, wherein the at least one free-radical film-forming polymer is present in the composition in an amount ranging from 10% to 35% by weight relative to the total weight of the composition.

7. The composition according to claim 1, wherein the at least one free-radical film-forming polymer is a vinyl polymer.

8. The composition according to claim 1, wherein the at least one free-radical film-forming polymer is an acrylic polymer.

9. The composition according to claim 1, wherein the at least one free-radical film-forming polymer results from the polymerization of monomers with ethylenic unsaturation having at least one acid group, esters of said monomers with ethylenic unsaturation having at least one acid group, and amides of said monomers with ethylenic unsaturation having at least one acid group.

10. The composition according to claim 1, wherein the at least one polymer having at least one fatty chain is present in the composition in an amount ranging from 0.5% to 40% by weight relative to the total weight of the composition.

11. The composition according to claim 10, wherein the at least one polymer having at least one fatty chain is present in the composition in an amount ranging from 2% to 30% by weight relative to the total weight of the composition.

12. The composition according to claim 11, wherein the at least one polymer having at least one fatty chain is present in the composition in an amount ranging from 5% to 20% by weight relative to the total weight of the composition.

13. The composition according to claim 1, further comprising at least one adjuvant chosen from colorants, pigments, pearlescent agents, lacquers, anti-UV agents, thickening agents, surfactants, waxes, silicones, perfumes, preservatives, moisturizing agents, and active agents.

14. The composition according to claim 1, wherein the composition is in a form chosen from a nail varnish, a nail care composition, an eyeliner, a mascara, a foundation, an eyeshadow, a blusher, a lipstick, a concealer, a make-up product for the body, a care composition, an anti-sun composition, and a self-tanning composition.

15. The composition according to claim 14, wherein the make-up product for the body is chosen from compositions for temporary and semi-permanent tattooing.

16. A method for at least one of cosmetically treating and applying make-up to a keratinous material, comprising
applying to the keratinous material an effective amount of a composition comprising an aqueous dispersion of particles of at least one free-radical film-forming polymer and
an aqueous dispersion of at least one polymer having at least one fatty chain,
wherein the at least one polymer having at least one fatty chain has a length in terms of oil ranging from 5% to 90%, and
when the free-radical film-forming polymer/polymer having at least one fatty chain weight ratio is less than 1.5:1, the at least one polymer having at least one fatty chain has a length in terms of oil of less than 50%;
when the free-radical film-forming polymer/polymer having at least one fatty chain weight ratio is greater than or equal to 1.5:1, the at least one free-radical film-forming polymer has a glass transition temperature of less than or equal to 30° C.

17. A method for producing a film comprising,
applying to a keratinous material a composition comprising an aqueous dispersion of particles of at least one free-radical film-forming polymer and
an aqueous dispersion of at least one polymer having at least one fatty chain,
wherein the at least one polymer having at least one fatty chain has a length in terms of oil ranging from 5% to 90%, and
when the free-radical film-forming polymer/polymer having at least one fatty chain weight ratio is less than 1.5:1, the at least one polymer having at least one fatty chain has a length in terms of oil of less than 50%;
when the free-radical film-forming polymer/polymer having at least one fatty chain weight ratio is greater than or equal to 1.5:1, the at least one free-radical film-forming polymer has a glass transition temperature of less than or equal to 30° C.,
wherein a film is formed on the keratinous material.

18. The method according to claim 17, wherein the at least one polymer having at least one fatty chain is an alkyd resin.

19. A method of plasticizing at least one free radical film-forming polymer comprising,
including an aqueous dispersion of at least one polymer having at least one fatty chain in a composition comprising an aqueous dispersion of particles of at least one free-radical film-forming polymer
wherein the at least one polymer having at least one fatty chain has a length in terms of oil ranging from 5% to 90%, and
when the free-radical film-forming polymer/polymer having at least one fatty chain weight ratio is less than 1.5: 1, the at least one polymer having at least one fatty chain has a length in terms of oil of less than 50%;
when the free-radical film-forming polymer/polymer having at least one fatty chain weight ratio is greater than or equal to 1.5, the at least one free-radical film-forming polymer has a glass transition temperature of less than or equal to 30° C.;
wherein the aqueous dispersion of at least one polymer having at least one fatty chain is a plasticizing agent for the at least one free-radical film-forming polymer.

20. The method according to claim 19, wherein at least one polymer having at least one fatty chain is an alkyd resin.
